(19) United States
(12) Patent Application Publication
(10) Pub. No.: US 2007/0005014 A1
(43) Pub. Date: Jan. 4, 2007

(54) Disposable safety syringe

(76) Inventors: Yu-Yueh Lin, Taipei (TW); Kun-Jung Yang, Jungli City (TW)

Correspondence Address: LOWE HAUPTMAN BERNER, LLP, 1700 DIAGONAL ROAD, SUITE 300, ALEXANDRIA, VA, 22314, US

(21) Appl. No.: 11/153,348

(22) Filed: Jun. 16, 2005

(51) Int. Cl. A61M 5/00

(52) U.S. Cl. 604/110; 604/187

(57) ABSTRACT

A disposable safety syringe consists of a needle, a connecting part, a barrel and a piston. The connecting part is inserted inside an inner tube of the barrel so as to prevent solution therein from leaking. The needle is disposed on a tubular bearing on front surface of the connecting part. The piston is removable—being pushed forwards and pulled backwards. By pushing the piston forwards, the solution passes the connecting part and the needle to be injected into a patient. The device is featured on that a pivotal piece with certain thickness is integrated and connected with a rear-end surface of the connecting part by a connecting piece. The connecting piece acts as a pivot. A round insertion hole with certain diameter is arranged on the center of the pivotal piece. A projecting post having a conic head with a hook on top side thereof, corresponding to the insertion hole, is disposed on a front-end surface of the piston. After finishing injection, the piston is pushed continuously so as to make the conic head insert through the insertion hole of the pivotal piece and hook with the pivotal piece. Thus when pulled the piston backwards, the connecting part is moved by the pivotal piece on one side and both the connecting part and the needle retracts into the barrel and are inclined so as to achieve safety use of the syringe.

### BACKGROUND OF THE INVENTION

The present invention relates to a disposable safety syringe, especially to a kind of syringe that retracts a needle into a barrel after use and makes the needle slant.

A syringe is a common medical device, including a needle, a barrel and a piston. For sake of sanitary and safety, the syringe is disposed after use. There are various kinds of design of syringe. For example, the needle retracts into the barrel before being disposed so as to avoid stabs of people and spread of infectious diseases. Moreover, another type of syringe is to destroy the needle, which retracts into the barrel after use, by the piston inside the barrel. Furthermore, there are two types of syringe-interchangeable or fixed needle. The interchangeable type is encased on front end of the barrel and the needles with various diameter are changeable according to users' needs. While the fixed-type—the needle is inserted through an opening on rear end of the barrel (also the opening for accommodating the piston) and then is fixed on the front end of the barrel so as to make the needle tubing expose outside. It's difficult or inconvenient to replace the needle. However, conventional disposable syringes have more complicated structure thus increase difficulties and cost on mass production. It's also inconvenient to retract the needle or destroy the needle tubing. Due to lack of interchangeability of the needles, the cost is increased.

### SUMMARY OF THE INVENTION

It is therefore a primary object of the present invention to provide a disposable safety syringe composed by a needle, a connecting part, a barrel and a piston. The connecting part is inserted inside an inner tube of the barrel so as to prevent solution therein from leaking. The needle is encased on a tubular bearing on front surface of the connecting part while a pivotal piece with certain thickness is integrated and connected with a rear-end surface of the connecting part by a connecting piece. The connecting piece acts as a pivot so as to make the pivotal piece attach on or depart from the rear-end surface of the connecting part. A round insertion hole with certain diameter is arranged on the center of the pivotal piece. A projecting post having a conic head with a hook on top side thereof, corresponding to the insertion hole, is disposed on a front-end surface of the piston. After finishing injection, the piston is pushed continuously so as to make the conic head insert through the insertion hole of the pivotal piece and hook with the pivotal piece. Thus when pulled the piston backwards, the connecting part is moved by the pivotal piece on one side and both the connecting part and the needle retracts into the barrel and are inclined so as to achieve safety use of the syringe.

It is another object of the present invention to provide a disposable safety syringe that has a connecting part inserted and fixed on front end of an inner tube of the barrel. A tubing is disposed on front-end surface of the connecting part for connection with the needle. This syringe is suitable for both fixed needle or interchangeable needle. Thus the convenience and application of the safety syringe are increased.

### BRIEF DESCRIPTION OF THE DRAWINGS

FIG. 1 is a perspective view of an embodiment with a fixed needle in accordance with the present invention;

FIG. 2 is an explosive view of the embodiment shown in FIG. 1;

FIG. 3 is a lateral sectional view of the embodiment shown in FIG. 1;

FIG. 4 is a lateral sectional view of the embodiment shown in FIG. 3 while the piston leans against the connecting part;

FIG. 5 is an enlarged view of a connecting part with a pivotal piece attached thereof in accordance with the present invention;

FIG. 6 is a lateral sectional view of the connecting part shown in FIG. 5;

FIG. 7 is an enlarged view of a connecting part with a pivotal piece departed thereof shown in FIG. 5;

FIG. 8 is a lateral sectional view of the connecting part shown in FIG. 7;

FIG. 9 is an enlarged view of another embodiment of the connecting piece on the connecting part;

FIG. 10 is a lateral sectional view of the connecting part shown in FIG. 9;

FIG. 11 is a lateral sectional view of an embodiment shown in FIG. 3 while sucking the solution;

FIG. 12 is a lateral sectional view of an embodiment in accordance with the present invention while injecting the solution;

FIG. 13 is a lateral sectional view of an embodiment showing the piston in connection with the pivotal piece;

FIG. 14 is a lateral sectional view of an embodiment showing the retraction of the connecting part;

FIG. 15 is a lateral sectional view of an embodiment in FIG. 14 showing the destruction of the needle tubing;

FIG. 16 is a lateral sectional view of an embodiment in FIG. 15 showing the breaking of the piston shaft;

FIG. 17 is an explosive view of another embodiment with an interchangeable needle in accordance with the present invention;

FIG. 18 is a lateral sectional view of the embodiment in FIG. 16 showing the separation of the needle;

FIG. 19 is a lateral sectional view of the embodiment in FIG. 16;

FIG. 20 is a lateral sectional view of an embodiment in FIG. 18 showing the retraction of the connecting part;

FIG. 21 is a lateral sectional view of an embodiment in FIG. 19 showing the destruction of the needle tubing.

### DETAILED DESCRIPTION OF THE PREFFERED EMBODIMENT

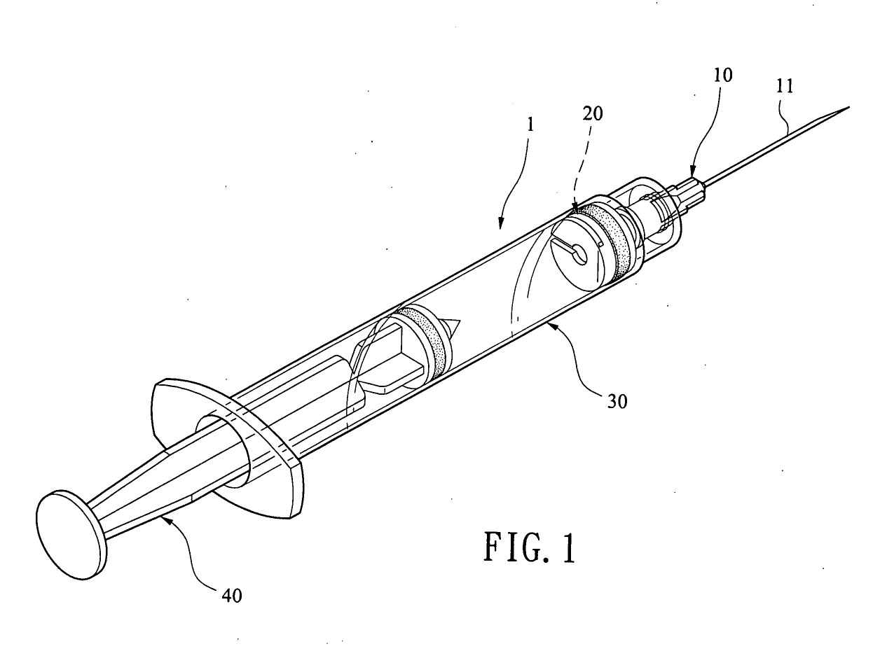

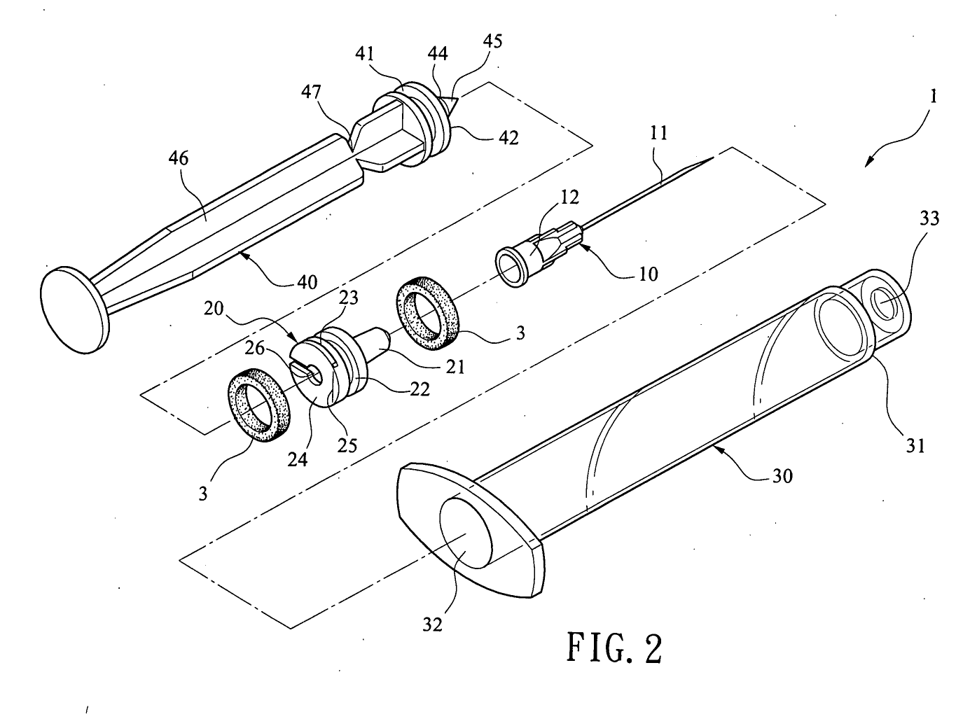

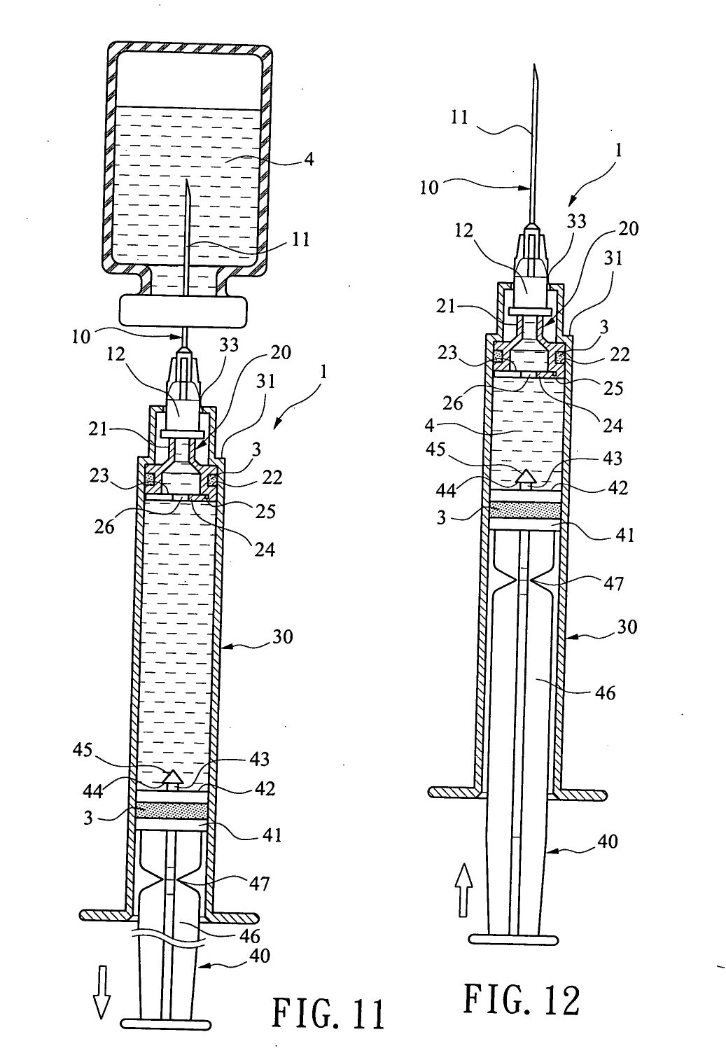

Refer from FIG. 1 to FIG. 4, a safety syringe 1 in accordance with the present invention is composed by a needle 10, a connecting part 20, a barrel 30 and a piston 40. The connecting part 20 is inserted into and fixed on a front-end area 31 so as to prevent solution therein from leaking. The needle 10 consisting of a needle tubing 11 and a needle bed 12 is encased on a tubular bearing 21 on front surface of the connecting part 20 and then is inserted through a rear-end opening 32 of the barrel 30 so that when the connecting part 20 is localized on the front-end area 31 of the barrel 30, the needle tubing 20 exposes outside a front-end opening 33 of the barrel 30. This is so-called fixed needle that is unchangeable after assembling or when being used. The piston 40 is moved forwards and backwards inside the barrel 30 while a circular slot 41 for insertion of a sealing ring 3 to form a stopper on rear end for solution is disposed on outer surface of the front end thereof. In usage, the piston 40 is pulled backwards so as to make solution 4 inside the bottle flow into the barrel 30, as shown in FIG. 11. Then push the piston 40 forwards to make the solution 4 pass the connecting part 20 and the needle 10, ready to use, as shown in FIG. 12.

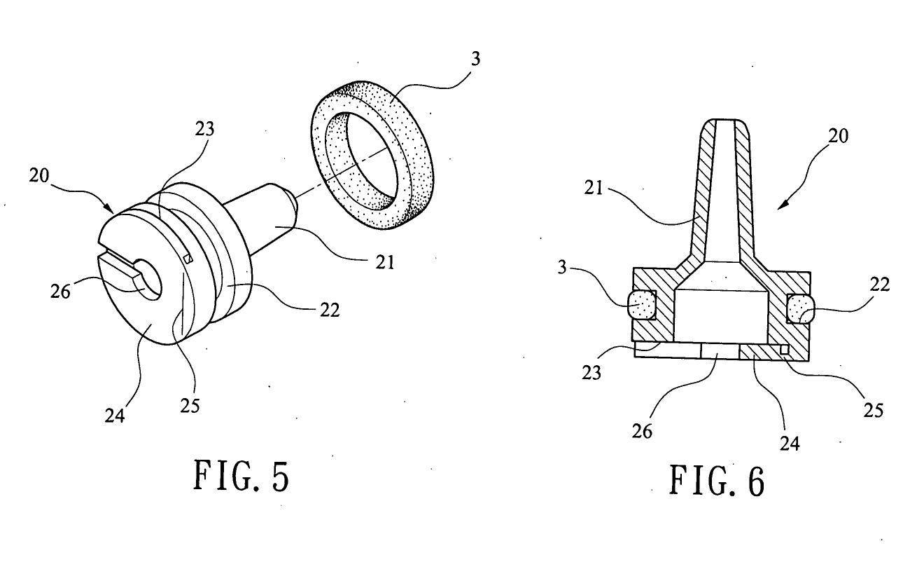

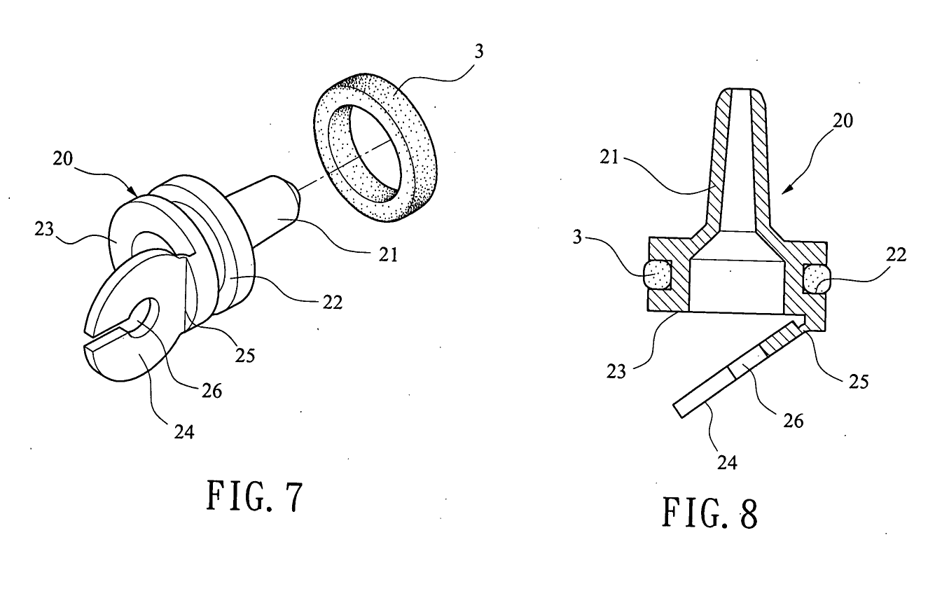

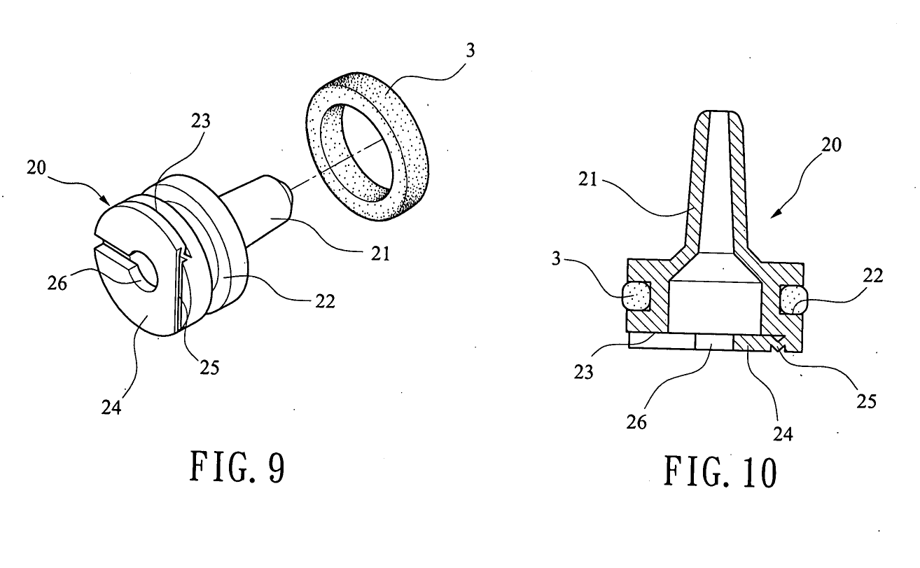

Refer from FIG. 5 to FIG. 8, the connecting part 20 is an integrated part with an inner channel. A tubular bearing 21 with smaller diameter is arranged on surface of front end of the connecting part 20 for connection with the needle bed 12 of the needle 10 while at least one circular slot 22 for accommodating a sealing ring 3 is formed on outer surface of the connecting part 20. The diameter of the connecting part 20 corresponds to the inner diameter of the barrel 30 so as to make itself be inserted and sealed on the front-end area 31 of the barrel 30. A pivotal piece 24 with certain thickness is pivoted on a rear-end surface 23 of the connecting part 20 by a connecting piece 25 on part of the edge thereof. The connecting piece 25 acts like a pivot of the pivotal piece 24 so that the pivotal piece 24 can attach on the rear-end surface 23 of the connecting part 20, as shown in FIG. 5 & FIG. 6, or the pivotal piece 24 can be apart from the rear-end surface 23, as shown in FIG. 7 & FIG. 8. Furthermore, when the pivotal piece 24 attaches on the ear-end surface 23 of the connecting part 20, the pivotal piece 24 is smoothly covering on the outer surface of the ear-end surface 23, as shown in FIG. 5 & FIG. 6, this is the preferred status. Moreover, the connecting piece 25 can be designed with elasticity and V-shaped cross section so that supporting elasticity thereof is increased, as shown in FIG. 9 & FIG. 10.Thus the pivotal piece 24 is attached elastically on the ear-end surface 23 of the connecting part 20 without departing from it unless being pulled by an external force. In addition, a round insertion hole 26 with certain diameter that connects to the inner channel of the connecting part 20 is arranged on the center of the pivotal piece 24.

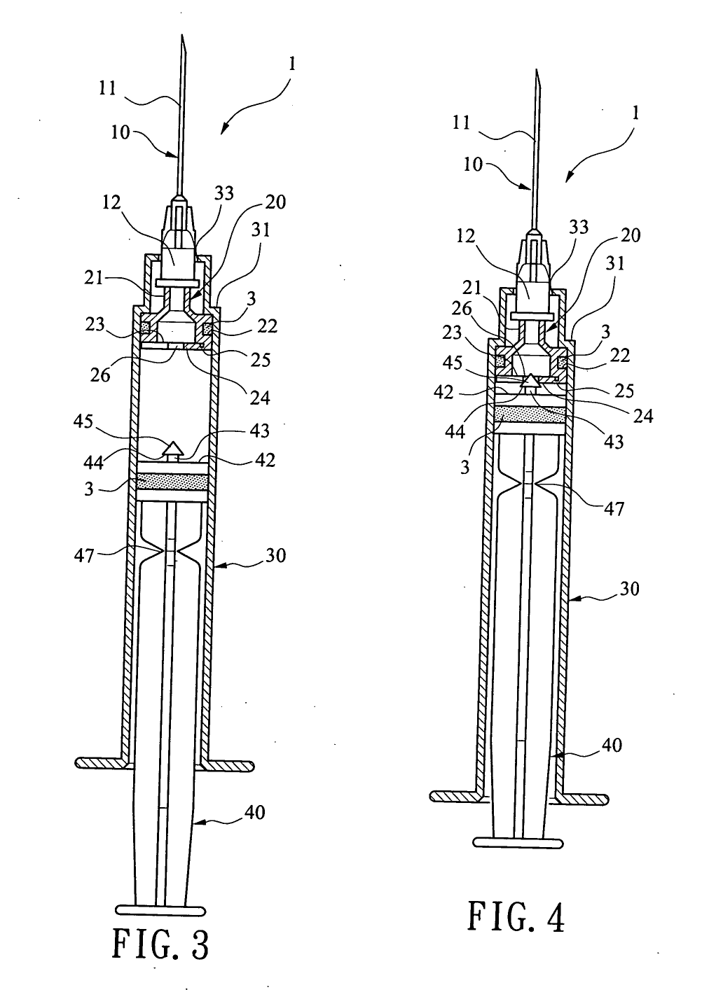

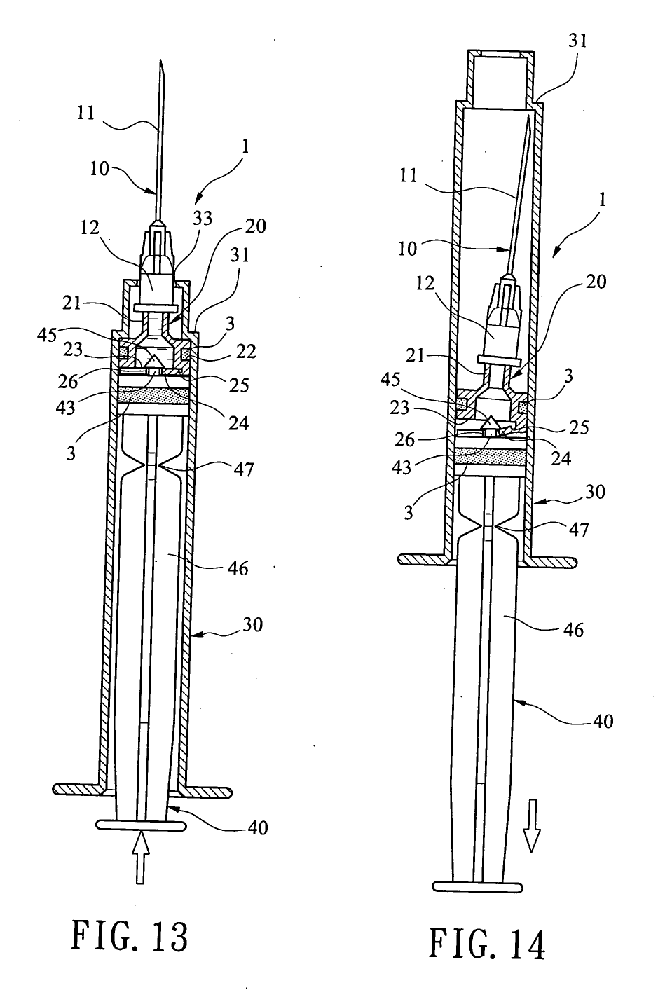

Refer to FIG. 3 & FIG. 4, a projecting post 43 with certain height is disposed on a front-end surface 42 of the piston 40. A conic head 45 with a hook 44 is installed on top of the projecting post 43. The size of the conic head 45 corresponds to the diameter of the insertion hole 26. When the piston 40 is pushed forwards so as to make the conic head 45 against on the insertion hole 26 of the pivotal piece 24, the injection procedure is finished. As shown in FIG. 4, most of the conic head 45 inserts into the insertion hole 26 while the hook 44 hasn't passed through the e insertion hole 26. Then, deal with the safety syringe 1 before disposition. Continuingly push the piston 40 for forcing the conic head 45 moving forward so that the hook 44 inserts into and hooks with the insertion hole 26, as shown in FIG. 13. Later, pull the piston 40 in reverse direction, then the pivotal piece 24 leaves the rear-end surface 23 and slants in an angle as shown in FIG. 7, FIG. 8, & FIG. 14. By the connecting piece 25, the connecting part 20 and the needle 10 are continuingly moving backwards and retracting into the barrel 30 so as to achieve safety use of syringes, as shown in FIG. 14. Now pull out the piston 40 and break a piston shaft 46 by a breakage 47 thereof so as to prevent the piston 40 from pushing forwards and causing the needle 10 exposed outside accidentally, as shown in FIG. 16.

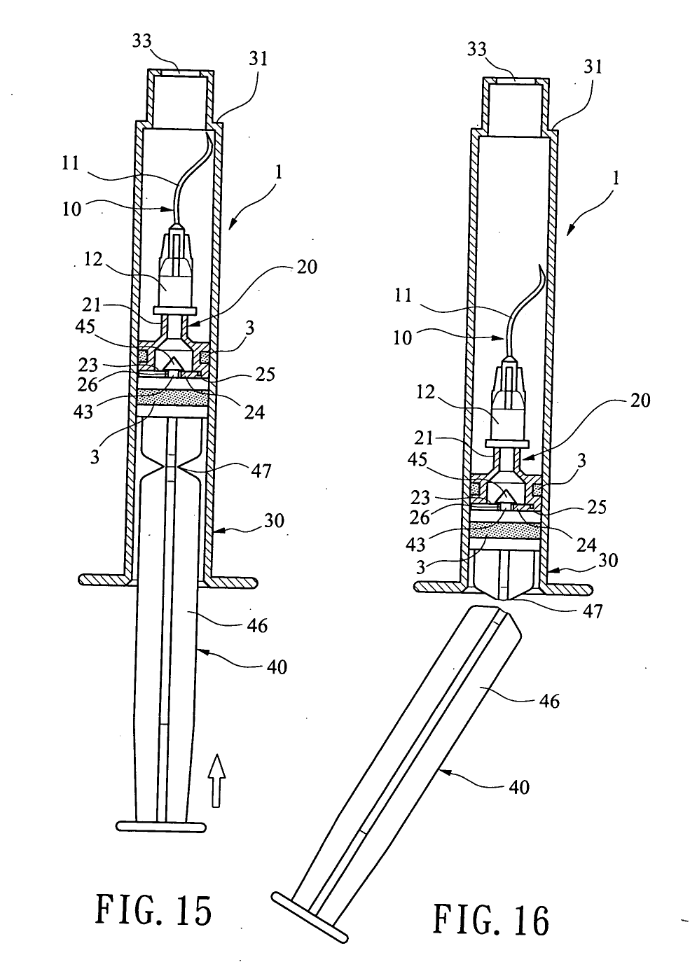

Because the connecting piece 25 between the pivotal piece 24 and the rear-end surface 23 of the connecting part 20 is located near the edge so that the drawing force from the connecting piece 25 to the connecting part 20 is towards the center of the connecting part 20 and a torque of the connecting part 20 is formed. Thus the connecting part 20 slants during the retraction procedure and the needle 10 on the front end of the connecting part 20 also inclines, as shown in FIG. 14. Now the tip of the needle tubing 11 is against an inner flange on the front-end area 31 of the barrel 30, the piston 40 is pushed forwards so as to make the needle tubing 11 bend and crashed inside the barrel 30, as shown in FIG. 15. Then the piston shaft 46 is broken by the breakage 47.

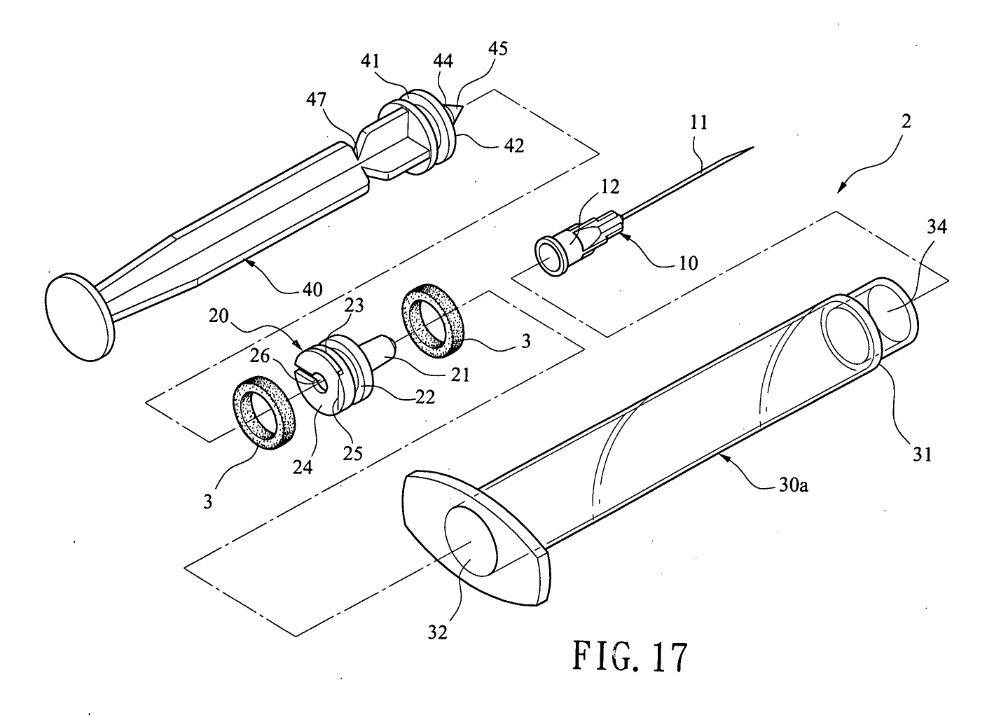

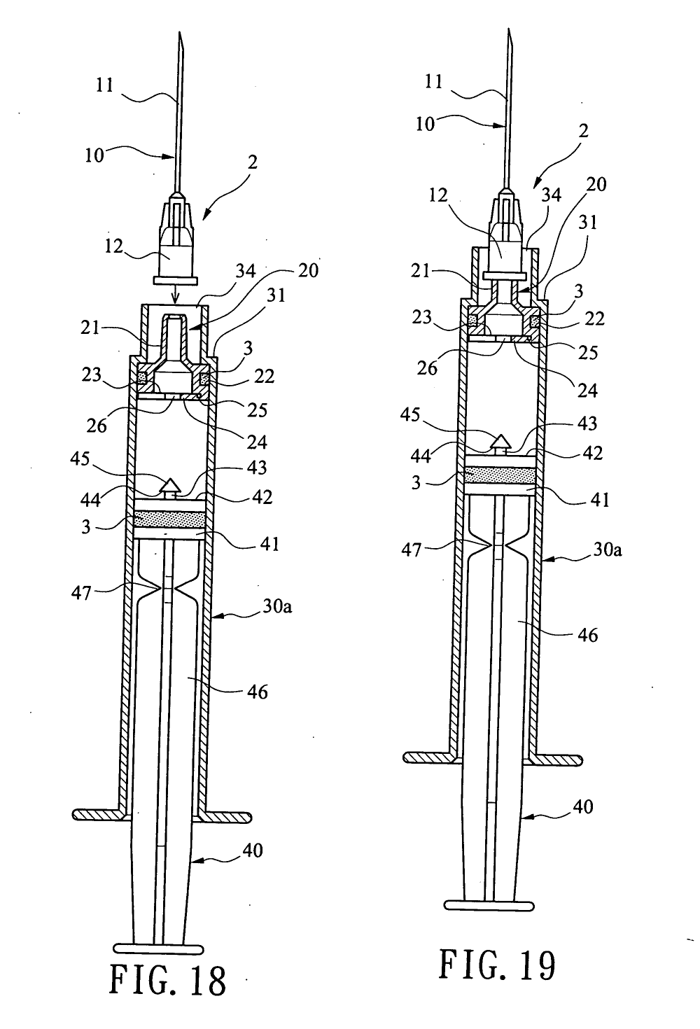

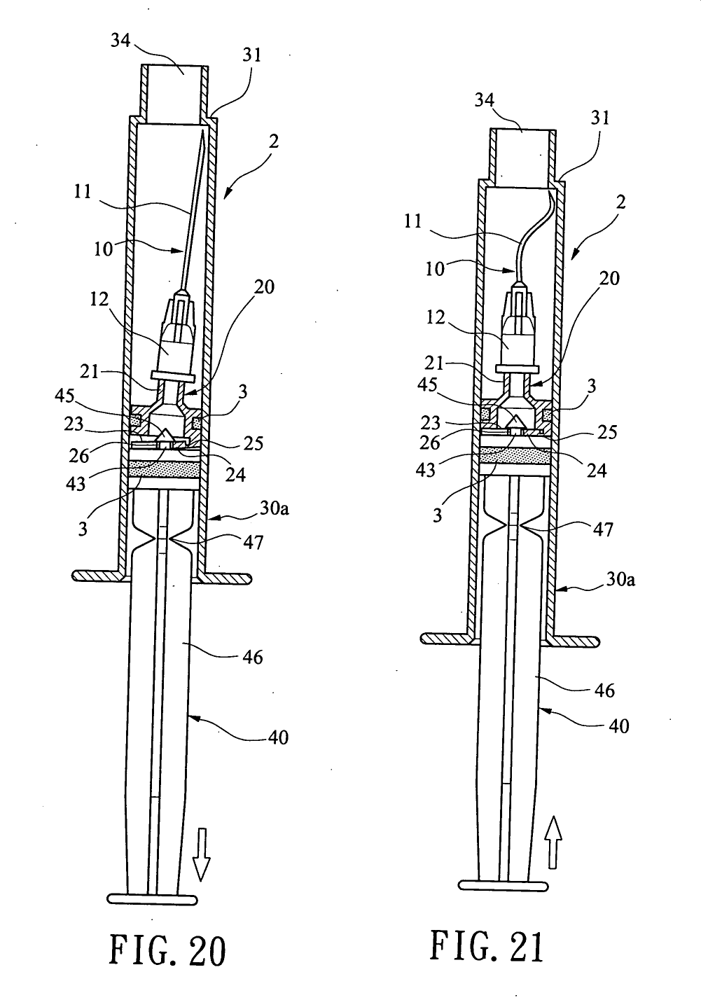

Refer to FIG. 17, FIG. 18, FIG. 19, FIG. 20 & FIG. 21, the safety Syringe 2 has the same parts with the safety syringe 1 with the same functions, as shown in FIG. 20 & FIG. 21. The difference between these two embodiments is in that a front-end opening 34 is arranged on a barrel 30a of the safety syringe 2. The diameter of the front-end opening 34 is larger than that of the front-end opening 33 of the barrel 30 of the safety syringe 1. When assembling, the connecting part 20 is inserted into the barrel 30 through the rear-end opening 32 and then is located and fixed on the front-end area 31 of the inner tube of the barrel 30a so as to form a front stopper for the solution. The needle 10 is encased on a tubular bearing 21 on front-end surface of the connecting part 20 and a needle tubing 12 is exposing outside the front-end opening 34 of the barrel 30a. This is so-called an interchangeable needle that is interchangeable according to user's needs.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A disposable safety syringe comprising a needle, a connecting part, a barrel and a piston, wherein:
the needle having a needle tubing and a needle bed while the needle bed is disposed on a tubular bearing on front-end surface of the connecting part;
the connecting part is inserted into front end area and fixed thereof; the connecting part as an integrated part with an inner channel having a tubular bearing with smaller diameter arranged on surface of front end thereof for connection with the needle, at least one circular slot for accommodating a sealing ring formed on outer surface thereof, and a pivotal piece with certain thickness pivoted on a rear-end surface thereof by a connecting piece on part of the edge thereof; the connecting piece acts as a pivot of the pivotal piece so as to make the pivotal piece attach on or depart from the rear-end surface of the connecting part; a round insertion hole that connects to the inner channel of the connecting part is arranged on the center of the pivotal piece;
a piston moving forwards and backwards inside the barrel and having a circular slot for insertion of the sealing ring disposed on outer surface of the front end thereof and a projecting post disposed on a front-end surface thereof; the projecting post with certain height corresponds to the insertion hole of the pivotal piece and having a conic head with a hook on topside thereof;
thereby when the piston is pushed forwards to make the conic head against the insertion hole, the piston is further pushed to force the conic head passing through and hooked with the insertion hole; when the piston is pulled backwards, the pivotal piece leaves the rear-end surface and inclines and then by drawing of the connecting piece, the connecting part and the needle retracts inside the barrel.

2. The device as claimed in claim 1, wherein the needle is disposed on the tubular bearing on front-end surface of the connecting part and then inserted into the barrel through a rear-end opening of the barrel so that the needle tubing exposes outside a front end opening of the barrel when the connecting part is located on front end area of the inner tube of the barrel.

3. The device as claimed in claim 1, wherein the connecting part is inserted into the barrel through a rear-end opening of the barrel and is located on front end area of the inner tube of the barrel, then the needle is arranged on the tubular bearing on front-end surface of the connecting part through a front-end opening of the barrel so that the needle tubing exposes outside the front end opening of the barrel.

4. The device as claimed in claim 1, wherein the pivotal piece is parallel to outer surface of the rear-end surface of the connecting part as a best status when the pivotal piece attaches on the rear-end surface of the connecting part.
